(19) United States
(12) Patent Application Publication
(10) Pub. No.: US 2013/0098396 A1
(43) Pub. Date: Apr. 25, 2013

(54) NOVEL 1,1,1,4,4,5,5,6,6,6-DECAFLUOROHEX-2-ENE ISOMER MIXTURES AND USES THEREOF

(71) Applicant: E I DU PONT DE NEMOURS AND COMPANY, Wilmington, DE (US)

(72) Inventors: Robert D. Lousenberg, Wilmington, DE (US); Xuehui Sun, Swedesboro, NJ (US); Joan Ellen Bartelt, Wilmington, DE (US)

(73) Assignee: E I DU PONT DE NEMOURS AND COMPANY, Wilmington, DE (US)

(21) Appl. No.: 13/646,893

(22) Filed: Oct. 8, 2012

Related U.S. Application Data
(60) Provisional application No. 61/549,069, filed on Oct. 19, 2011.

(51) Int. Cl. B08B 7/00; B05D 5/00; C11D 7/60; B08B 1/00; C07C 21/18; C07C 17/278

(52) U.S. Cl. 134/6; 570/136; 570/153; 510/461; 134/31; 134/42; 427/384; 427/127; 427/240

(57) ABSTRACT

Disclosed are compositions comprising unsaturated hydrofluorocarbons, an alkene with the formula of 1,1,1,4,4,5,5,6,6,6-decafluorohex-2-ene and its isomers (the “153-10 isomers”). The invention further relates to use of said compositions in methods to clean, degrease, deflux, dewater, deposit fluorolubricant, carrier fluid applications and heat transfer applications. The invention further relates to novel 153-10 isomer mixtures, their method of making and their use as cleaning compositions and in the methods listed above.

### BACKGROUND INFORMATION

1. Field of the Disclosure

This invention relates to a specialty fluid material for applications such as cleaning. The specialty fluid material compositions comprise unsaturated hydrofluorocarbons, an alkene with the formula of 1,1,1,4,4,5,5,6,6,6-decafluorohex-2-ene and its isomers (the “153-10 isomers”). The invention further relates to use of said cleaning compositions in methods to clean, degrease, deflux, dewater, deposit fluorolubricant, carrier fluid applications and heat transfer applications. The invention further relates to novel 153-10 isomer mixtures, their method of making and their use as cleaning compositions and in the methods listed above.

2. Description of the Related Art

Chlorofluorocarbon (CFC) compounds have been used extensively in the area of semiconductor manufacture to clean surfaces such as magnetic disk media. However, chlorine-containing compounds such as CFC compounds are considered to be detrimental to the Earth's ozone layer. In addition, many of the hydrofluorocarbons used to replace CFC compounds have been found to contribute to global warming. Therefore, there is a need to identify new environmentally safe solvents for cleaning applications, such as removing residual flux, lubricant or oil contaminants, and particles. There is also a need for identification of new solvents for deposition of fluorolubricants and for drying or dewatering of substrates that have been processed in aqueous solutions.

The present invention provides new compositions comprising 153-10 isomer mixtures, and methods of manufacture of the isomers. These compositions have utility in many of the applications formerly served by CFC compounds. For example, the 153-10 isomer mixtures can be used as carrier fluid in lubricant deposition, for oxygen service cleaning, as drying and rinsing agents, as heat transfer fluids, as cleaning agents in high-voltage dielectrics, as cleaning solvents, for particulate and ionic removal, for light, medium, and heavy soil removal, in silicone deposition and tube swelling, in defluxing, for precision cleaning, and for optics cleaning. The 153-10 isomers is envisioned to have uses similar to the DuPont Vertrel® specialty fluid, which has HFC-43-10mee as its main component (also known as 2,3-dihydrodecafluoropentane). HFC-43-10 has a boiling point of 54° C. HFC-43-10 is non-ozone depleting but has global warming potential (GWP) of 1400, relative to carbon dioxide. On the other hand, the 153-10 isomer mixture is a hydrofluoroolefin (HFO) and is predicted to have a significantly lower GWP due to its unsaturation.

The compositions of the present invention possess some or all of the desired properties of little or no environmental impact, ability to dissolve oils, greases or lubricants (in particular fluorine-containing lubricants), non-flammability, and ability to dissolve surfactant compounds used in methods for drying or dewatering.

### SUMMARY

In one embodiment, this invention relates to a composition comprising at least one or more of:
(A) a first isomer 1,1,4,4,5,5,5-heptafluoro-3-(trifluoromethyl)pent-1-ene;
(B) a second isomer 1,1,1,2,2,3,5,6,6,6,-decafluorohex-3-ene; and
(C) mixtures thereof.

In another embodiment, the present invention relates to the above compositions further comprising a third isomer 1,1,1,4,4,5,5,6,6,6-decafluorohex-2-ene.

This invention also relates to a process for preparation of compositions described above, comprising:
(A) contacting 1,1,1,4,4,4-hexafluorobut-2-ene with 1,1,2,2-tetrafluoroethylene, to provide said composition mixed with other reaction products; and
(B) optionally separating said other reaction products from said composition;
   wherein said contacting step takes place in the presence of a catalytically effective amount of:
   (i) MCl_{5-y}F_{y}, wherein M=Sb, Nb, Ta, Mo, and y=0 to 5;
   (ii) SbCl₃₋ₓFₓ (x=0 to 3);
   (iii) aluminum halide composition having a bulk formula of AlX_{y}F_{3-y}, wherein the average value of y is 0 to 3, wherein X is Cl or Br;
   (iv) BF₃;
   (v) FeX₃ wherein X is selected from the group consisting of Cl and F, and FeX₃ supported on carbon;
   (vi) AsF₃; and/or
   (vii) M′Cl_{4-z}F_{z}, wherein M′=Sn, Ti, Zr, Hf; z=0 to 4.

In yet another embodiment, said contacting step is undertaken in the temperature range of from about −50° C. to about +20° C.

This invention further relates to a method for removing residue from a surface of an article comprising:
(A) contacting said surface with a composition described above, and
(B) recovering the surface from said composition.

In another embodiment, this invention further relates to a method for depositing a fluorolubricant on a surface comprising:
(A) combining a fluorolubricant and a solvent, said solvent comprising said composition described above to form a lubricant-solvent combination;
(B) contacting said combination of lubricant-solvent with the surface; and
(C) evaporating said solvent from said surface to form a fluorolubricant coating on said surface.

The foregoing general description and the following detailed description are exemplary and explanatory only and are not restrictive of the invention, as defined in the appended claims.

### DRAWINGS

FIG. 1. Structures and IUPAC names of NMR suggested 153-10 isomers from Example 3.

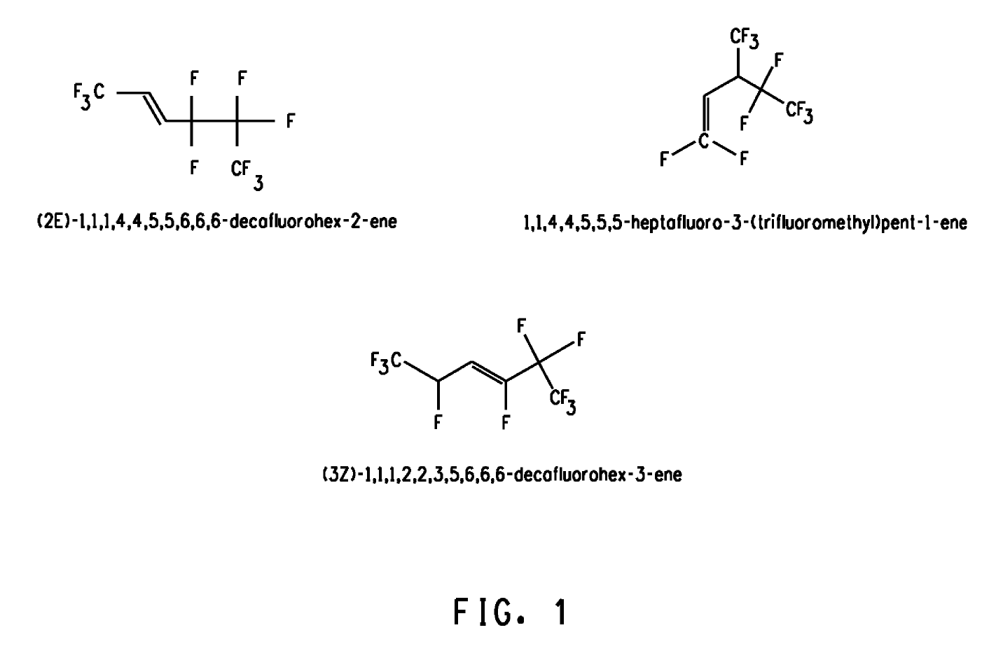

FIG. 2. Structures and IUPAC names of NMR suggested 153-10 isomers in Example 4.

FIG. 3. ¹H NMR of main product 153-10 isomer mixture from Example 3.

FIG. 4. ¹⁹F NMR of main product 153-10 isomer mixture from Example 3.

FIG. 5. ¹⁹F NMR of main product 153-10 isomer mixture from Example 3.

FIG. 6. ¹⁹F NMR of main product 153-10 isomer mixture in Example 4.

FIG. 7. ¹⁹F NMR of main product 153-10 isomer mixture from Example 4.

FIG. 8. ¹⁹F NMR of main product 153-10 isomer mixture from Example 4.

### DETAILED DESCRIPTION

Disclosed herein are compositions comprising at least one or more of (A) a first isomer 1,1,4,4,5,5,5-heptafluoro-3-(trifluoromethyl)pent-1-ene; (B) a second isomer 1,1,1,2,2,3,5,6,6,6,-decafluorohex-3-ene; and (C) mixtures thereof. Also disclosed herein are compositions comprising a third isomer 1,1,1,4,4,5,5,6,6,6-decafluorohex-2-ene) in addition to the above disclosed compositions.

In one embodiment of the above compositions, said first isomer is in the range of from about 0.5% to about 99.5% of the total weight of the first isomer and the second isomer if the third isomer is not present and first isomer, second isomer, and third isomer if the third isomer is present. Stated another way, the first isomer content can be about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 30.5%, 31%, 31.5%, 32%, 32.5%, 33%, 33.5%, 34%, 34.5%, 35%, 35.5%, 36%, 36.5%, 37%, 37.5%, 38%, 38.5%, 39%, 39.5%, 40%, 40.5%, 41%, 41.5%, 42%, 42.5%, 43%, 43.5%, 44%, 44.5%, 45%, 45.5%, 46%, 46.5%, 47%, 47.5%, 48%, 48.5%, 49%, 49.5%, 50%, 50.5%, 51%, 51.5%, 52%, 52.5%, 53%, 53.5%, 54%, 54.5%, 55%, 55.5%, 56%, 56.5%, 57%, 57.5%, 58%, 58.5%, 59%, 59.5%, 60%, 60.5%, 61%, 61.5%, 62%, 62.5%, 63%, 63.5%, 64%, 64.5%, 65%, 65.5%, 66%, 66.5%, 67%, 67.5%, 68%, 68.5%, 69%, 69.5%, 70%, 70.5%, 71%, 71.5%, 72%, 72.5%, 73%, 73.5%, 74%, 74.5%, 75%, 75.5%, 76%, 76.5%, 77%, 77.5%, 78%, 78.5%, 79%, 79.5%, 80%, 80.5%, 81%, 81.5%, 82%, 82.5%, 83%, 83.5%, 84%, 84.5%, 85%, 85.5%, 86%, 86.5%, 87%, 87.5%, 88%, 88.5%, 89%, 89.5%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, and about 99.5%.

In one embodiment of the above compositions, said third isomer is in the range of from about 0.5% to about 99.5% of the total weight of the first isomer and the third isomer if the second isomer is not present and first isomer, second isomer, and third isomer if the second isomer is present. Stated another way, the third isomer content can be about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 30.5%, 31%, 31.5%, 32%, 32.5%, 33%, 33.5%, 34%, 34.5%, 35%, 35.5%, 36%, 36.5%, 37%, 37.5%, 38%, 38.5%, 39%, 39.5%, 40%, 40.5%, 41%, 41.5%, 42%, 42.5%, 43%, 43.5%, 44%, 44.5%, 45%, 45.5%, 46%, 46.5%, 47%, 47.5%, 48%, 48.5%, 49%, 49.5%, 50%, 50.5%, 51%, 51.5%, 52%, 52.5%, 53%, 53.5%, 54%, 54.5%, 55%, 55.5%, 56%, 56.5%, 57%, 57.5%, 58%, 58.5%, 59%, 59.5%, 60%, 60.5%, 61%, 61.5%, 62%, 62.5%, 63%, 63.5%, 64%, 64.5%, 65%, 65.5%, 66%, 66.5%, 67%, 67.5%, 68%, 68.5%, 69%, 69.5%, 70%, 70.5%, 71%, 71.5%, 72%, 72.5%, 73%, 73.5%, 74%, 74.5%, 75%, 75.5%, 76%, 76.5%, 77%, 77.5%, 78%, 78.5%, 79%, 79.5%, 80%, 80.5%, 81%, 81.5%, 82%, 82.5%, 83%, 83.5%, 84%, 84.5%, 85%, 85.5%, 86%, 86.5%, 87%, 87.5%, 88%, 88.5%, 89%, 89.5%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, and about 99.5%.

In one embodiment of the above compositions, said second isomer is not present. In another embodiment, said second isomer is present in the range of from about 0.5% to about 99.5% of the total weight of the three isomers. Stated another way, the third isomer content can be about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 30.5%, 31%, 31.5%, 32%, 32.5%, 33%, 33.5%, 34%, 34.5%, 35%, 35.5%, 36%, 36.5%, 37%, 37.5%, 38%, 38.5%, 39%, 39.5%, 40%, 40.5%, 41%, 41.5%, 42%, 42.5%, 43%, 43.5%, 44%, 44.5%, 45%, 45.5%, 46%, 46.5%, 47%, 47.5%, 48%, 48.5%, 49%, 49.5%, 50%, 50.5%, 51%, 51.5%, 52%, 52.5%, 53%, 53.5%, 54%, 54.5%, 55%, 55.5%, 56%, 56.5%, 57%, 57.5%, 58%, 58.5%, 59%, 59.5%, 60%, 60.5%, 61%, 61.5%, 62%, 62.5%, 63%, 63.5%, 64%, 64.5%, 65%, 65.5%, 66%, 66.5%, 67%, 67.5%, 68%, 68.5%, 69%, 69.5%, 70%, 70.5%, 71%, 71.5%, 72%, 72.5%, 73%, 73.5%, 74%, 74.5%, 75%, 75.5%, 76%, 76.5%, 77%, 77.5%, 78%, 78.5%, 79%, 79.5%, 80%, 80.5%, 81%, 81.5%, 82%, 82.5%, 83%, 83.5%, 84%, 84.5%, 85%, 85.5%, 86%, 86.5%, 87%, 87.5%, 88%, 88.5%, 89%, 89.5%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, and about 99.5%.

In another embodiment of the above compositions, said first isomer is in the range of from about 40% to about 60%; said second isomer is in the range of from about 0 to about 15%; and said third isomer is in the range of from about 40 to about 50%, of the total weight of said first isomer, said second isomer, and said third isomer within the composition.

In yet another embodiment, the composition comprises said first isomer and said third isomer; wherein said first isomer is about 55% and said third isomer is about 45% by weight of the total weight of said first isomer and said third isomer; and wherein the weight content of said second isomer is substantially zero.

The 153-10 isomer mixture described herein is a potential drop-in replacement for HFC-43-10, the current component of Vertrel®, one of the reasons being that the 153-10 isomer mixture is anticipated to have a much lower GWP (up to 100× lower) than HFC-43-10 and may meet market demand for a similar boiling point specialty fluid with a lower GWP.

In one embodiment, the 153-10 isomer mixture described herein was prepared from a low-temperature reaction of cis- and/or trans-1,1,1,4,4,4-hexafluoro-2-butene with tetrafluoroethylene and a Lewis acid catalyst such as SbF₅. Cis-1,1,1,4,4,4-hexafluoro-2-butene is a current leading candidate for next generation foaming expansion agents (FEA) while trans-1,1,1,4,4,4-hexafluoro-2-butene is a leading candidate for next generation fire extinguishing (FE) agents.

In one embodiment of the present invention, the reaction of 1,1,1,4,4,4-hexafluoro-2-butene with TFE produces a mixture of two major and one minor 153-10 isomers. The major isomers are (2E)-1,1,1,4,4,5,5,6,6,6-decafluorohex-2-ene (trans-F13E) and 1,1,4,4,5,5,5-heptafluoro-3-(trifluoromethyl)pent-1-ene. The minor isomer is (3Z)-1,1,1,2,2,3,5,6,6,6-decafluorohex-3-ene. The three isomers have similar boiling points with a narrow boiling point range of 52-54° C.

In one embodiment, this invention also relates to a process for preparation of composition of comprising the first isomer, the second isomer, and the third isomer (the 153-10 isomers), comprising:
(A) contacting 1,1,1,4,4,4-hexafluorobut-2-ene with 1,1,2,2-tetrafluoroethylene in presence of a catalyst, to provide said composition mixed with other reaction products; and
(B) optionally separating said other reaction products from said composition.

In one embodiment, a suitable catalyst is SbF₅. In one embodiment, the reactor is chilled to in the temperature range of from about −60° C. to about 0° C. Stated another way, the initial reactor temperature can be about −60, −59, −58, −57, −56, −55, −54, −53, −52, −51, −50, −49, −48, −47, −46, −45, −44, −43, −42, −41, −40, −39, −38, −37, −36, −35, −34, −33, −32, −31, −30, −29, −28, −27, −26, −25, −24, −23, −22, −21, −20, −19, −18, −17, −16, −15, −14, −13, −12, −11, −10, −9, −8, −7, −6, −5, −4, −3, −2, −2, −1, and about 0° C. In another embodiment, the reactor vessel is warmed up from the initial temperature, generally to room temperature.

It is to be noted that the above reaction will generally form the three 153-10 isomers described previously as the first isomer, the second isomer, and the third isomer. In addition to the above isomers, the final product can have the unreacted 1,1,1,4,4,4-hexafluoro-2-butene, 173-14 isomers and possibly other products from ancillary reactions.

In one embodiment, the crude mixture of the 153-10 isomers is purified by distillation, which removes remaining trans 1,1,1,4,4,4-hexafluoro-2-butene, and higher TFE analogues such as tetradecafluoro-4-octene and/or other 173-14 isomers. In one embodiment, a first fraction of trans 1,1,1,4,4,4-hexafluoro-2-butene isomers was isolated at about 8° C. to 15° C. Thus, the first fraction could be isolated at about 8, 8.5, 9, 9.5, 10, 10.5, 11, 11.5, 12, 12.5, 13, 13.5, 14, 14.5, and about 15° C. Clearly, the isolation can occur over the entire range or a portion of the range disclosed herein and the temperatures intermediate to the specific temperatures pointed out herein are within the scope of the range disclosed herein.

In another embodiment, the second main fraction comprising 153-10 isomers is obtained at 45° C. to 55° C. Thus, the second main fraction can be obtained at 45, 45.5, 46, 46.5, 47, 47.5, 48, 48.5, 49, 49.5, 50, 50.5, 51, 51.5, 52, 52.5, 53, 53.5, 54, 54.5, and about 55° C. Clearly, the separation can occur over the entire range or a portion of the range disclosed herein and the temperatures intermediate to the specific temperatures pointed out herein are within the scope of the range disclosed herein.

In one embodiment, the main fraction of distillation comprising the 153-10 isomer mixtures is separated from water that was carried over in the distillation, dried over anhydrous magnesium sulfate. In another embodiment, the main fraction is dried over anhydrous sodium sulfate.

In another embodiment, disclosed herein are novel methods of using a composition described above.

Many aspects and embodiments have been described above and are merely exemplary and not limiting. After reading this specification, skilled artisans appreciate that other aspects and embodiments are possible without departing from the scope of the invention.

Other features and benefits of any one or more of the embodiments will be apparent from the following detailed description, and from the claims.

Before addressing details of embodiments described below, some terms are defined or clarified.

### Lewis Acid Catalysts

The above reaction for preparing 153-10 isomers is accomplished using Lewis acid catalysts. In one embodiment, the catalyst is SbF₅ or aluminum chlorofluoride.

Suitable catalysts which may be used for the preparation of the 153-10 isomers when carried out in the liquid phase include AlF₃, BF₃, FeX₃ where X is selected from the group consisting of Cl and F, FeX₃ supported on carbon, SbCl₃₋ₓFₓ (x=0 to 3), AsF₃, MCl_{5-y}F_{y} (M=Sb, Nb, Ta, Mo; x=0 to 5), M′Cl_{4-z}F_{z} (M′=Sn, Ti, Zr, Hf; z=0 to 4).

In one embodiment, the catalysts is at least one of the following:
(i) MCl_{5-y}F_{y}, wherein M=Sb, Nb, Ta, Mo, and y=0 to 5;
(ii) SbCl₃₋ₓFₓ (x=0 to 3);
(iii) aluminum halide composition having a bulk formula of AlX_{y}F_{3-y}, wherein the average value of y is 0 to 3, wherein X is Cl or Br;
(iv) BF₃;
(v) FeX₃ wherein X is selected from the group consisting of Cl and F, and FeX₃ supported on carbon;
(vi) AsF₃; and/or
(vii) M′Cl_{4-z}F_{z}, wherein M′=Sn, Ti, Zr, Hf; z=0 to 4, and
   wherein said contacting step is undertaken in the temperature range of from about −50° C. to about +20° C.

A suitable catalyst includes aluminum chlorofluoride (ACF), wherein the fluoride is from about 9.7% to 72.8% of the total halide content. It includes compounds such as aluminum dichlorofluoride (AlCl₂F) and aluminum chlorodifluoride (AlClF₂). In some catalyst compositions, the ACF catalyst can also be defined by the formula AlClₓF₃₋ₓ, wherein x=0.05-0.3. U.S. Pat. No. 3,158,593 describes a preparation of ACF.

In one embodiment, the present compositions may further comprise a propellant. Aerosol propellant may assist in delivering the present composition from a storage container to a surface in the form of an aerosol. Aerosol propellant is optionally included in the present composition in up to about 25 weight percent of the total composition. Representative aerosol propellants comprise air, nitrogen, carbon dioxide, difluoromethane (CF₂H₂, HFC-32), trifluoromethane (CF₃H, HFC-23), difluoroethane (CHF₂CH₃, HFC-152a), trifluoroethane (CH₃CF₃, HFC-143a; or CHF₂CH₂F, HFC-143), tetrafluoroethane (CF₃CH₂F, HFC-134a; or CF₂HCF₂H, HFC-134), pentafluoroethane (CF₃CF₂H, HFC-125), 1,3,3,3-tetrafluoro-1-propene (HFO-1234ze), 2,3,3,3-tetrafluoro-1-propene (HFO-1234yf), 1,2,3,3,3-pentafluoropropene (HFO-1225ye), 1,1,3,3,3-pentafluoropropene (HFO-1225ze) and hydrocarbons, such as propane, butanes, or pentanes, or dimethyl ether.

In another embodiment, the present compositions may further comprise at least one surfactant. The surfactants of the present invention include all surfactants known in the art for dewatering or drying of substrates. Representative surfactants include alkyl phosphate amine salts (such as a 1:1 salt of 2-ethylhexyl amine and isooctyl phosphate); ethoxylated alcohols, mercaptans or alkylphenols; quaternary ammonium salts of alkyl phosphates (with fluoroalkyl groups on either the ammonium or phosphate groups); and mono- or di-alkyl phosphates of fluorinated amines. Additional fluorinated surfactant compounds are described in U.S. Pat. No. 5,908,822, incorporated herein by reference.

The amount of surfactant included in the dewatering compositions of the present invention can vary widely depending on the particular drying application in which said composition will be used, but is readily apparent to those skilled in the art. In one embodiment, the amount of surfactant dissolved in the unsaturated fluorinated ether solvent is not greater than about 1 weight percent, based on the total weight of the surfactant/solvent composition. In another embodiment, larger amounts of surfactant can be used, if after treatment with the composition, the substrate being dried is thereafter treated with solvent containing either no or minimal surfactant. In one embodiment, the amount of surfactant is at least about 50 parts per million (ppm, on a weight basis). In another embodiment, the amount of surfactant is from about 100 to about 5000 ppm. In yet another embodiment, the amount of surfactant used is from about 200 to about 2000 ppm based on the total weight of the dewatering composition.

Optionally, other additives may be included in the present compositions comprising solvents and surfactants for use in dewatering. Such additives include compounds having antistatic properties; the ability to dissipate static charge from non-conductive substrates such as glass and silica. Use of an antistatic additive in the dewatering compositions of the present invention may be necessary to prevent spots and stains when drying water or aqueous solutions from electrically non-conductive parts such as glass lenses and mirrors. Most unsaturated fluoroether solvents of the present invention also have utility as dielectric fluids, i.e., they are poor conductors of electric current and do not easily dissipate static charge. Boiling and general circulation of dewatering compositions in conventional drying and cleaning equipment can create static charge, particularly in the latter stages of the drying process where most of the water has been removed from a substrate. Such static charge collects on non-conductive surfaces of the substrate and prevents the release of water from the surface. The residual water dries in place resulting in undesirable spots and stains on the substrate. Static charge remaining on substrates can bring out impurities from the cleaning process or can attract impurities such as lint from the air, which results in unacceptable cleaning performance. In one embodiment, desirable antistatic additives are polar compounds, which are soluble in the present unsaturated fluorinated ether solvent and result in an increase in the conductivity of the unsaturated fluorinated ether solvent resulting in dissipation of static charge from a substrate. In another embodiment, the antistatic additives have a normal boiling point near that of the unsaturated fluorinated ether solvent and have minimal to no solubility in water. In yet another embodiment, the antistatic additives have a solubility in water of less than about 0.5 weight percent. In one embodiment, the solubility of antistatic agent is at least 0.5 weight percent in unsaturated fluorinated ether solvent. In one embodiment, the antistatic additive is nitromethane (CH₃NO₂).

In one embodiment, the present dewatering composition containing an antistatic additive is effective in both the dewatering and drying and rinse steps of a method to dewater or dry a substrate as described below.

Another embodiment relates to a method for dewatering or drying a substrate comprising:
(A) contacting the surface with a composition comprising at least one or more of (I) a first isomer 1,1,4,4,5,5,5-heptafluoro-3-(trifluoromethyl)pent-1-ene; (II) a second isomer 1,1,1,2,2,3,5,6,6,6,-decafluorohex-3-ene; and (III) mixtures thereof, and
(B) recovering the dewatered substrate from the composition.

Optionally, the above composition may also further comprise a third isomer 1,1,1,4,4,5,5,6,6,6-decafluorohex-2-ene.

Many industries use aqueous compositions for the surface treatment of metals, ceramics, glasses, and plastics. Cleaning, plating, and deposition of coatings are often carried out in aqueous media and are usually followed by a step in which residual water is removed. Hot air drying, centrifugal drying, and solvent-based water displacement are methods used to remove such residual water.

While hydrofluorocarbons (HFCs) have been proposed as replacements for the previously used CFC solvents in drying or dewatering applications, many HFCs have limited solvency for water. The use of surfactant, which assists in removal of water from substrates is therefore necessary in many drying or dewatering methods. Hydrophobic surfactants have been added to dewatering or drying solvents to displace water from substrates.

The primary function of the dewatering or drying solvent (unsaturated fluorinated ether solvent) in a dewatering or drying composition is to reduce the amount of water on the surface of a substrate being dried. The primary function of the surfactant is to displace any remaining water from the surface of the substrate. When the unsaturated fluorinated ether solvent and surfactant are combined, a highly effective displacement drying composition is attained.

In one embodiment, the surfactant for dewatering and drying is soluble to at least 1 weight percent based on the total solvent/surfactant composition weight.

In one embodiment, the dewatering or drying method of the present disclosure is very effective in displacing water from a broad range of substrates including metals, such as tungsten, copper, gold, beryllium, stainless steel, aluminum alloys, brass and the like; from glasses and ceramic surfaces, such as glass, sapphire, borosilicate glass, alumina, silica such as silicon wafers used in electronic circuits, fired alumina and the like; and from plastics such as polyolefin (“Alathon”, Rynite®, “Tenite”), polyvinylchloride, polystyrene (Styron), polytetrafluoroethylene (Teflon®), tetrafluoroethylene-ethylene copolymers (Tefzel®), polyvinylidenefluoride (“Kynar”), ionomers (Surlyn®), acrylonitrile-butadiene-styrene polymers (Kralac®), phenol-formaldehyde copolymers, cellulosic (“Ethocel”), epoxy resins, polyacetal (Delrin®), poly(p-phenylene oxide) (Noryl®), polyetherketone (“Ultrapek”), polyetheretherketone (“Victrex”), poly(butylene terephthalate) (“Valox”), polyarylate (Arylon®), liquid crystal polymer, polyimide (Vespel®), polyetherimides (“Ultem”), polyamideimides (“Torlon”), poly(p-phenylene sulfide) (“Rython”), polysulfone (“Udel”), and polyaryl sulfone (“Rydel”). In another embodiment, the compositions for use in the present dewatering or drying method are compatible with elastomers.

In one embodiment, the disclosure is directed to a process for removing at least a portion of water from, i.e., dewatering, the surface of a wetted substrate, which comprises contacting the substrate with the aforementioned dewatering composition, and then removing the substrate from contact with the dewatering composition. In one embodiment, water originally bound to the surface of the substrate is displaced by solvent and/or surfactant and leaves with the dewatering composition. By “at least a portion of water” is meant at least about 75 weight percent of water at the surface of a substrate is removed per immersion cycle. By “immersion cycle” is meant one cycle involving at least a step wherein substrate is immersed in the present dewatering composition. Optionally, minimal amounts of surfactant remaining adhered to the substrate can be further removed by contacting the substrate with surfactant-free halocarbon solvent. Holding the article in the solvent vapor or refluxing solvent will further decrease the presence of surfactant remaining on the substrate. Removal of solvent adhering to the surface of the substrate is effected by evaporation. Evaporation of solvent at atmospheric or subatmospheric pressures can be employed and temperatures above and below the boiling point of the halocarbon solvent can be used.

Methods of contacting the substrate with dewatering composition are not critical and can vary widely. For example, the substrate can be immersed in the composition, or the substrate can be sprayed with the composition using conventional equipment. Complete immersion of the substrate is preferred as it generally insures contact between the composition and all exposed surfaces of the substrate. However, any other method, which can easily provide such complete contact may be used.

The time period over which substrate and dewatering composition are contacted can vary widely. Usually, the contacting time is up to about 5 minutes, however, longer times may be used if desired. In one embodiment of the dewatering process, the contacting time is from about 1 second to about 5 minutes. In another embodiment, the contacting time of the dewatering process is from about 15 seconds to about 4 minutes.

Contacting temperatures can also vary widely depending on the boiling point of the composition. In general, the contacting temperature is equal to or less than the composition's normal boiling point.

In one embodiment, the compositions of the present disclosure may further contain a co-solvent. Such co-solvents are desirable where the present compositions are employed in cleaning conventional process residue from substrates, e.g., removing soldering fluxes and degreasing mechanical components comprising substrates of the present invention. Such co-solvents include alcohols (such as methanol, ethanol, isopropanol), ethers (such as diethyl ether, methyl tertiary-butyl ether), ketones (such as acetone), esters (such as ethyl acetate, methyl dodecanoate, isopropyl myristate and the dimethyl or diisobutyl esters of succinic, glutaric or adipic acids or mixtures thereof), ether alcohols (such as propylene glycol monopropyl ether, dipropylene glycol monobutyl ether, and tripropylene glycol monomethyl ether), and hydrocarbons (such as pentane, cyclopentane, hexane, cyclohexane, heptane, octane), and hydrochlorocarbons (such as trans-1,2-dichloroethylene). When such a co-solvent is employed with the present composition for substrate dewatering or cleaning, it may be present in an amount of from about 1 weight percent to about 50 weight percent based on the weight of the overall composition.

In cleaning apparatuses, including vapor degreasing and vapor defluxing equipment, compositions may be lost during operation through leaks in shaft seals, hose connections, soldered joints and broken lines. In addition, the working composition may be released to the atmosphere during maintenance procedures on equipment. If the composition is not a pure component, the composition may change when leaked or discharged to the atmosphere from the equipment, which may cause the composition remaining in the equipment to exhibit unacceptable performance. Accordingly, it is desirable to use as a cleaning composition comprising a single unsaturated fluorinated ether.

Another embodiment relates to a method of cleaning a surface comprising:
(A) contacting the surface with a composition comprising at least one or more of (I) a first isomer 1,1,4,4,5,5,5-heptafluoro-3-(trifluoromethyl)pent-1-ene; (II) a second isomer 1,1,1,2,2,3,5,6,6,6,-decafluorohex-3-ene; and (III) mixtures thereof, and
(B) recovering the surface from the composition.

Optionally, the above composition may also further comprise a third isomer 1,1,1,4,4,5,5,6,6,6-decafluorohex-2-ene.

In one embodiment, the compositions of the present disclosure are useful as cleaning compositions, cleaning agents, deposition solvents and as dewatering or drying solvents. For proper operation in use, microelectronic components must be cleaned of flux residues, oils and greases, and particulates that may contaminate the surfaces after completion of manufacture. In another embodiment, the present disclosure relates to a process for removing residue from a surface or substrate comprising contacting the surface or substrate with a cleaning composition or cleaning agent of the present invention and, optionally, recovering the surface or substrate substantially free of residue from the cleaning composition or cleaning agent.

In yet another embodiment, the present disclosure relates to a method for cleaning surfaces by removing contaminants from the surface. The method for removing contaminants from a surface comprises contacting the surface having contaminants with a cleaning composition of the present invention to solubilized the contaminants and, optionally, recovering the surface from the cleaning composition. The surface is then substantially free of contaminants.

As stated previously, the contaminants or residues that may be removed by the present method include, but are not limited to oils and greases, flux residues, and particulate contaminants.

In one embodiment of the method, the contacting may be accomplished by spraying, flushing, wiping with a substrate e.g., wiping cloth or paper, that has the cleaning composition incorporated in or on it. In another embodiment of the method, the contacting may be accomplished by dipping or immersing the disk in a bath of the cleaning composition.

In one embodiment of the method, the recovering is by removing the surface that has been contacted from the cleaning composition bath (in a similar manner as described for the method for depositing an a fluorolubricant on a surface as described below). In another embodiment of the method, the recovering is by allowing the cleaning composition that has been sprayed, flushed, or wiped on the disk to drain away. Additionally, any residual cleaning composition that may be left behind after the completion of the previous steps may be evaporated in a manner similar to that for the deposition method as well.

The method for cleaning a surface may be applied to the same types of surfaces as the method for deposition as described below. Semiconductor surfaces or magnetic media disks of silica, glass, metal or metal oxide, or carbon may have contaminants removed by the method. In the method described above, contaminant may be removed from a disk by contacting the disk with the cleaning composition and recovering the disk from the cleaning composition.

In yet another embodiment, the present method also provides methods of removing contaminants from a product, part, component, substrate, or any other article or portion thereof by contacting the article with a cleaning composition of the present invention. For the purposes of convenience, the term “article” is used herein to refer to all such products, parts, components, substrates, and the like and is further intended to refer to any surface or portion thereof. Furthermore, the term “contaminant” is intended to refer to any unwanted material or substance present on the article, even if such substance is placed on the article intentionally. For example, in the manufacture of semiconductor devices it is common to deposit a photoresist material onto a substrate to form a mask for the etching operation and to subsequently remove the photoresist material from the substrate. The term “contaminant” as used herein is intended to cover and encompass such a photo resist material. Hydrocarbon based oils and greases and dioctylphthalate are examples of the contaminants that may be found on the carbon coated disks.

In one embodiment, the present method comprises contacting the article with a cleaning composition of the invention, in a vapor degreasing and solvent cleaning method. In one such embodiment, vapor degreasing and solvent cleaning methods consist of exposing an article, preferably at room temperature, to the vapors of a boiling cleaning composition. Vapors condensing on the object have the advantage of providing a relatively clean, distilled cleaning composition to wash away grease or other contamination. Such processes thus have an additional advantage in that final evaporation of the present cleaning composition from the object leaves behind relatively little residue as compared to the case where the object is simply washed in liquid cleaning composition.

In another embodiment, for applications in which the article includes contaminants that are difficult to remove, the present methods involve raising the temperature of the cleaning composition above ambient or to any other temperature that is effective in such application to substantially improve the cleaning action of the cleaning composition. In one such embodiment, such processes are also generally used for large volume assembly line operations where the cleaning of the article, particularly metal parts and assemblies, must be done efficiently and quickly.

In one embodiment, the cleaning methods of the present invention comprise immersing the article to be cleaned in liquid cleaning composition at an elevated temperature. In another embodiment, the cleaning methods of the present invention comprise immersing the article to be cleaned in liquid cleaning composition at about the boiling point of the cleaning composition. In one such embodiment, this step removes a substantial amount of the target contaminant from the article. In yet another embodiment, this step removes a major portion of the target contaminant from the article. In one embodiment, this step is then followed by immersing the article in freshly distilled cleaning composition, which is at a temperature below the temperature of the liquid cleaning composition in the preceding immersion step. In one such embodiment, the freshly distilled cleaning composition is at about ambient or room temperature. In yet another embodiment, the method also includes the step of then contacting the article with relatively hot vapor of the cleaning composition, by exposing the article to vapors rising from the hot/boiling cleaning composition associated with the first mentioned immersion step. In one such embodiment, this results in condensation of the cleaning composition vapor on the article. In certain preferred embodiments, the article may be sprayed with distilled cleaning composition before final rinsing.

It is contemplated that numerous varieties and types of vapor degreasing equipment are adaptable for use in connection with the present methods. One example of such equipment and its operation is disclosed by U.S. Pat. No. 3,085,918, which is incorporated herein by reference. The equipment disclosed therein includes a boiling sump for containing a cleaning composition, a clean sump for containing distilled cleaning composition, a water separator, and other ancillary equipment.

The present cleaning methods may also comprise cold cleaning in which the contaminated article is either immersed in the fluid cleaning composition of the present invention under ambient or room temperature conditions or wiped under such conditions with rags or similar objects soaked in the cleaning composition.

Another embodiment relates to a method of depositing a fluorolubricant on a surface comprising: combining a fluorolubricant and a solvent, said solvent comprising at least one or more of (I) a first isomer 1,1,4,4,5,5,5-heptafluoro-3-(trifluoromethyl)pent-1-ene; (II) a second isomer 1,1,1,2,2,3,5,6,6,6,-decafluorohex-3-ene; and (III) mixtures thereof, to form a lubricant-solvent combination; contacting the combination of lubricant-solvent with the surface; and evaporating the solvent from the surface to form a fluorolubricant coating on the surface. Optionally, the above composition may also further comprise a third isomer 1,1,1,4,4,5,5,6,6,6-decafluorohex-2-ene.

The most advanced, highest recording densities and lowest cost method of storing digital information involves writing and reading magnetic flux patterns from rotating disks coated with magnetic materials. A magnetic layer, where information is stored in the form of bits, is sputtered onto a metallic support structure. Next an overcoat, usually a carbon-based material, is placed on top of the magnetic layer for protection and finally a lubricant is applied to the overcoat. A read-write head flies above the lubricant and the information is exchanged between the head and the magnetic layer. In a relentless attempt to increase the efficiency of information transfer, hard drive manufacturers have reduced the distance between the head and the magnetic layer, or fly-height, to less than 100 Angstroms.

Invariably, during normal disk drive application, the head and the disk surface will make contact. To reduce wear on the disk, from both sliding and flying contacts, it must be lubricated.

Fluorolubricants are widely used as lubricants in the magnetic disk drive industry to decrease the friction between the head and disk, that is, reduce the wear and therefore minimize the possibility of disk failure.

There is a need in the industry for improved methods for deposition of fluorolubricants. The use of certain solvents, such as CFC-113 and PFC-5060, has been regulated due to their impact on the environment. Therefore, solvents that will be used in this application should consider environmental impact. Also, such solvent must dissolve the fluorolubricant and form a substantially uniform or uniform coating of fluorolubricant. Additionally, existing solvents have been found to require higher fluorolubricant concentrations to produce a given thickness coating and produce irregularities in uniformity of the fluorolubricant coating.

In one embodiment, the fluorolubricants of the present disclosure comprise perfluoropolyether (PFPE) compounds, or lubricant comprising X-1P®, which is a phosphazene-containing disk lubricant. These perfluoropolyether compounds are sometimes referred to as perfluoroalkylethers (PFAE) or perfluoropolyalkylethers (PFPAE). These PFPE compounds range from simple perfluorinated ether polymers to functionalized perfluorinated ether polymers. PFPE compounds of different varieties that may be useful as fluorolubricant in the present invention are available from several sources. In another embodiment, useful fluorolubricants for the present inventive method include but are not limited to Krytox® GLP 100, GLP 105 or GLP 160 (E. I. du Pont de Nemours & Co., Fluoroproducts, Wilmington, Del., 19898, USA); Fomblin® Z-Dol 2000, 2500 or 4000, Z-Tetraol, or Fomblin® AM 2001 or AM 3001 (sold by Solvay Solexis S.p.A., Milan, Italy); Demnum™ LR-200 or S-65 (offered by Daikin America, Inc., Osaka, Japan); X-1P® (a partially fluorinated hexaphenoxy cyclotriphosphazene disk lubricant available from Quixtor Technologies Corporation, a subsidiary of Dow Chemical Co, Midland, Mich.); and mixtures thereof. The Krytox® lubricants are perfluoroalkylpolyethers having the general structure F(CF(CF₃)CF₂O)ₙ—CF₂CF₃, wherein n ranges from 10 to 60. The Fomblin® lubricants are functionalized perfluoropolyethers that range in molecular weight from 500 to 4000 atomic mass units and have general formula X—CF₂—O(CF₂—CF₂—O)ₚ—(CF₂O)_{q}—CF₂—X, wherein X may be —CH₂OH, CH₂(O—CH₂—CH₂)ₙOH, CH₂OCH₂CH(OH)CH₂OH or —CH₂O—CH₂-piperonyl. The Demnum™ oils are perfluoropolyether-based oils ranging in molecular weight from 2700 to 8400 atomic mass units. Additionally, new lubricants are being developed such as those from Moresco (Thailand) Co., Ltd, which may be useful in the present inventive method.

The fluorolubricants of the present invention may additionally comprise additives to improve the properties of the fluorolubricant. X-1P®, which may serve as the lubricant itself, is often added to other lower cost fluorolubricants in order to increase the durability of disk drives by passivating Lewis acid sites on the disk surface responsible for PFPE degradation.

Other common lubricant additives may be used in the fluorolubricants of the present inventive methods.

The fluorolubricants of the present invention may further comprise Z-DPA (Hitachi Global Storage Technologies, San Jose, Calif.), a PFPE terminated with dialkylamine end-groups. The nucleophilic end-groups serve the same purpose as X1P®, thus providing the same stability without any additive.

The surface on which the fluorolubricant may be deposited is any solid surface that may benefit from lubrication. Semiconductor materials such as silica disks, metal or metal oxide surfaces, vapor deposited carbon surfaces or glass surfaces are representative of the types of surfaces for which the methods of the present invention are useful. The present inventive method is particularly useful in coating magnetic media such as computer drive hard disks. In the manufacture of computer disks, the surface may be a glass, or aluminum substrate with layers of magnetic media that is also coated by vapor deposition with a thin (10-50 Angstrom) layer of amorphous hydrogenated or nitrogenated carbon. The fluorolubricant may be deposited on the surface disk indirectly by applying the fluorolubricant to the carbon layer of the disk.

The first step of combining the fluorolubricant and solvent may be accomplished in any suitable manner such as mixing in a suitable container such as a beaker or other container that may be used as a bath for the deposition method. The fluorolubricant concentration in the unsaturated fluorinated ether solvent may be from about 0.010 percent (wt/wt) to about 0.50 percent (wt/wt).

The step of contacting said combination of fluorolubricant and solvent with the surface may be accomplished in any manner appropriate for said surface (considering the size and shape of the surface). A hard drive disk must be supported in some manner such as with a mandrel or some other support that may fit through the hole in the center of the disk. The disk will thus be held vertically such that the plane of the disk is perpendicular to the solvent bath. The mandrel may have different shapes including but not limited to, a cylindrical bar, or a V-shaped bar. The mandrel shape will determine the area of contact with the disk. The mandrel may be constructed of any material strong enough to hold the disk, including but not limited to metal, metal alloy, plastic or glass. Additionally, a disk may be supported vertically upright in a woven basket or be clamped into a vertical position with 1 or more clamps on the outer edge. The support may be constructed of any material with the strength to hold the disk, such as metal, metal alloy, plastic or glass. However the disk is supported, the disk will be lowered into a container holding a bath of the fluorolubricant/solvent combination. The bath may be held at room temperature or be heated or cooled to temperatures ranging from about 0° C. to about 50° C.

Alternatively, the disk may be supported as described above and the bath may be raised to immerse the disk. In either case, the disk may then be removed from the bath (either by lowering the bath or by raising the disk). Excess fluorolubricant/solvent combination can be drained into the bath.

Either of the methods for contacting the fluorolubricant/solvent combination with the disk surface of either lowering the disk into a bath or raising a bath to immerse the disk are commonly referred to as dip coating. Other methods for contacting the disk with the fluorolubricant/solvent combination may be used in the present inventive method, including spraying or spin coating.

When the disk is removed from the bath, the disk will have a coating of fluorolubricant and some residual solvent (unsaturated fluorinated ether) on its surface. The residual solvent may be evaporated. Evaporation is usually performed at room temperature. However, other temperatures both above and below room temperature may be used as well for the evaporation step. Temperatures ranging from about 0° C. to about 100° C. may be used for evaporation.

The surface, or the disk if the surface is a disk, after completion of the coating method, will be left with a substantially uniform or uniform coating of fluorolubricant that is substantially free of solvent. The fluorolubricant may be applied to a thickness of less than about 300 nm, and alternately to a thickness of about 100 to about 300 nm.

A uniform fluorolubricant coating is desired for proper functioning of a disk and so areas of varying fluorolubricant thickness are undesirable on the surface of the disk. As more and more information is being stored on the same size disk, the read/write head must get closer and closer to the disk in order to function properly. If irregularities due to variation in coating thickness are present on the surface of the disk, the probability of contact of the head with these areas on the disk is much greater. While there is a desire to have enough fluorolubricant on the disk to flow into areas where it may be removed by head contact or other means, coating that is too thick may cause “smear,” a problem associated with the read/write head picking up excess fluorolubricant.

One specific coating thickness irregularity observed in the industry is that known as the “rabbit ears” effect. These irregularities are visually detected on the surface of the disk after deposition of the fluorolubricant using the existing solvent systems. When the disk is contacted with the solution of fluorolubricant in solvent and then removed from the solution, any points where the solution may accumulate and not drain readily develop drops of solution that do not readily drain off. One such point of drop formation is the contact point (or points) with the mandrel or other support device with the disk. When a V-shaped mandrel is used, there are two contact points at which the mandrel contacts the inside edge of the disk. When solution of fluorolubricant forms drops in these locations that do not drain off when removed from the bath, an area of greater thickness of fluorolubricant is created when the solvent evaporates. The two points of contact with the disk produces what is known as a “rabbit ears” effect, because the areas of greater fluorolubricant thickness produce a pattern resembling rabbit ears visually detectable on the disk surface.

When dip coating is used for depositing fluorolubricant on the surface, the pulling-up speed (speed at which the disk is removed from the bath), and the density of the fluorolubricant and the surface tension are relevant for determining the resulting film thickness of the fluorolubricant. Awareness of these parameters for obtaining the desired film thickness is required. Details on how these parameters effect coatings are given in, “Dip-Coating of Ultra-Thin Liquid Lubricant and its Control for Thin-Film Magnetic Hard Disks” in IEEE Transactions on Magnetics, vol. 31, no. 6, November 1995.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Unless otherwise defined, all technical and scientific terms used herein have the same meaning as commonly understood by one of ordinary skill in the art to which this invention belongs. Although methods and materials similar or equivalent to those described herein can be used in the practice or testing of embodiments of the present invention, suitable methods and materials are described below. All publications, patent applications, patents, and other references mentioned herein are incorporated by reference in their entirety, unless a particular passage is cited. In case of conflict, the present specification, including definitions, will control. In addition, the materials, methods, and examples are illustrative only and not intended to be limiting.

### Abbreviations and Legends

The common names of compounds are given on the left and their structures or other identifying characteristics are provided on the right in the two columns below:
CFC Chlorofluorocarbon
cis-1336m/z CF₃CH═CHCF₃ or 1,1,1,4,4,4-hexafluoro-2-butene
153-10 first isomer 1,1,4,4,5,5,5-heptafluoro-3-(trifluoromethyl)pent-1-ene or CF₃CF₂CH(CF₃)CH═CF₂
153-10 second isomer (3Z) 1,1,1,2,2,3,5,6,6,6,-decafluorohex-3-ene or CF₃CHFCH═CFCF₂CF₃
153-10 third isomer (2E) 1,1,1,4,4,5,5,6,6,6-decafluorohex-2-ene or CF₃CH═CHCF₂CF₂CF₃
HFC-173-14 1,1,1,2,2,3,3,6,6,7,7,8,8,8,-tetradecafluorooct-4-ene or n-C₃F₇CH═CH-n-C₃F₇
GWP Global Warming Potential
HFC-43-10mee 2,3-dihydrodecafluoropentane
TFE Tetrafluoroethylene
trans-1336m/z CF₃CH═CHCF₃ or 1,1,1,4,4,4-hexafluoro-2-butene
173-14 isomers C₈F₁₄H₂

### EXAMPLES

The concepts described herein will be further described in the following examples, which do not limit the scope of the invention described in the claims.

### Example 1

Example 1 demonstrates the reaction of trans 1,1,1,4,4,4-hexafluoro-2-butene with tetrafluoroethylene to make the 153-10 isomers in the presence of SbF₅ catalyst.

A 400-ml Hastelloy® shaker tube was charged with 8 g (0.037 mol) of SbF₅ as catalyst. The reactor was chilled to −45° C. and was twice evacuated and purged with N₂. At −45° C., 170 g (1 mol) of CF₃CH═CHCF₃ was added to the reactor under vacuum. Subsequently, 70 g (0.7 mol) of tetrafluoroethylene was added slowly in 10 g increments to the reactor. The contents in the reactor were stirred after all tetrafluoroethylene was added. The reactor was allowed to warm up to the room temperature without any external heating. Stirring was continued for additional 15 min after the reactor reached room temperature. Subsequently, the reactor was immediately chilled to −40° C. In the next step, 70 ml of phosphate buffer was slowly injected into the reactor. The pressure of the reactor decreased from 105 psig to 8 psig during the reaction while the temperature increased from −55° C. to 8° C. The product was vapor-transferred in a receiver cylinder and was analyzed by GC-MS. The data were reported by area percent of GC-MS plot. The analysis of liquid phase of the product showed the selectivity to 153-10 isomer is about 86% (see Table 1 below).

TABLE 1

| | | | GC-MS |
|---|---|---|---|
| Compound | Chemical Name | Tradename | area % |
| CF₃CH═CHCF₃ | 1,1,1,4,4,4-hexafluoro-2- | trans 1,1,1,4,4,4- | 18.2 |
| | butene | hexafluoro-2- | |
| | | butene/trans-1336mzz | |
| CF₃CH═CHCF₂CF₂CF₃ | 1,1,1,4,4,5,5,6,6,6- | 153-10 isomer | 30.3 |
| | octafluoro-2-hexene | | |
| CF₃CF₂CH(CF₃)CH═CF₂ | 1,1,4,4,5,5,5-heptafluoro- | 153-10 isomer | 34.1 |
| | 3-trifluoromethyl-1-petene | | |
| CF₃CHFCH═CFCF₂CF₃ | 1,1,1,2,2,3,5,6,6,6- | 153-10 isomer | 5.7 |
| | octafluoro-3-hexene | | |
| C₈F₁₄H₂ | | Three 173-14 | 11.6 |
| | | isomers | |

### Example 2

Example 2 demonstrates the reaction of trans 1,1,1,4,4,4-hexafluoro-2-butene with tetrafluoroethylene to make the 153-10 isomers in presence of aluminum chlorofluoride as catalyst.

A 400-ml Hastelloy® shaker tube was charged with 2 g of aluminum chlorofluoride as catalyst. The reactor was chilled to −10° C. and was twice evacuated and purged with N₂. At −10° C., 82.5 g (0.5 mol) of CF₃CH═CHCF₃ was added to the reactor under vacuum. Subsequently, 20 g (0.25 mol) of tetrafluoroethylene was added slowly to the reactor. The contents in the reactor were stirred after all tetrafluoroethylene was added. The reactor was allowed to warm up to the room temperature without any external heating. Stirring was continued for additional one hour after the reactor reached room temperature. In the next step, 20 ml water was slowly injected into the reactor. The pressure of the reactor decreased from 120 psig to 11 psig during the reaction. The product was vapor-transferred in a receiver cylinder and was analyzed by GC-MS. The data were reported by area percent of GC-MS plot. The analysis of liquid phase of the product showed the selectivity to 153-10 isomer is about 95% (see Table 2 below).

TABLE 2

| | | | GC- |
|---|---|---|---|
| | | Trade | MS |
| Compound | Chemical Name | Name | area % |
| CF₃CH═CHCF₃ | 1,1,1,4,4,4-hexafluoro-2- | trans- | 8.3 |
| | butene | 1336mzz | |
| CF₃CH═CHCF₂CF₂CF₃ | 1,1,1,4,4,5,5,6,6,6- | 153-10 | 40 |
| | decafluoro-2-hexene | isomer | |
| CF₃CF₂CH(CF₃)CH═CF₂ | 1,1,4,4,5,5,5-heptafluoro- | 153-10 | 47.6 |
| | 3-trifluoromethyl-1-petene | isomer | |
| CF₃CHFCH═CFCF₂CF₃ | 1,1,1,2,2,3,5,6,6,6- | 153-10 | 0.4 |
| | decafluoro-3-hexene | isomer | |
| C₈F₁₄H₂ | | two 173-14 | 3.8 |
| | | isomers | |

### Example 3

Example 3 demonstrates the distillation of crude 153-10 isomers from the reaction of trans 1,1,1,4,4,4-hexafluoro-2-butene with tetrafluoroethylene in presence of SbF₅ catalyst with a starting reaction temperature of −50° C.

The crude mixture of the 153-10 isomers was purified by distillation to remove remaining trans 1,1,1,4,4,4-hexafluoro-2-butene, and isolating the 153-10 isomers from higher TFE analogues (e.g. 4,5-dihydrotetradecafluoro-4-octene and/or other 173-14 isomers). The distillation apparatus consisted of a 1-L pot, a heating mantle with magnetic stirring; an 18-inch, Hastelloy®-packed and vacuum-jacketed column, a high-reflux-ratio (60:3 s/s) still-head with magnetic valve; and a condenser (starting at −15° C.). The crude products (6×˜240-g) were cannula-transferred to the dry ice-chilled still-pot. A first fraction (about 300 g) of trans 1,1,1,4,4,4-hexafluoro-2-butene isomers was isolated at about 9.4° C. to 12.8° C. A second main fraction (about 900 g) was obtained at 50° C. to 52° C. The heel (BP>75° C.) weighed about 170 g.

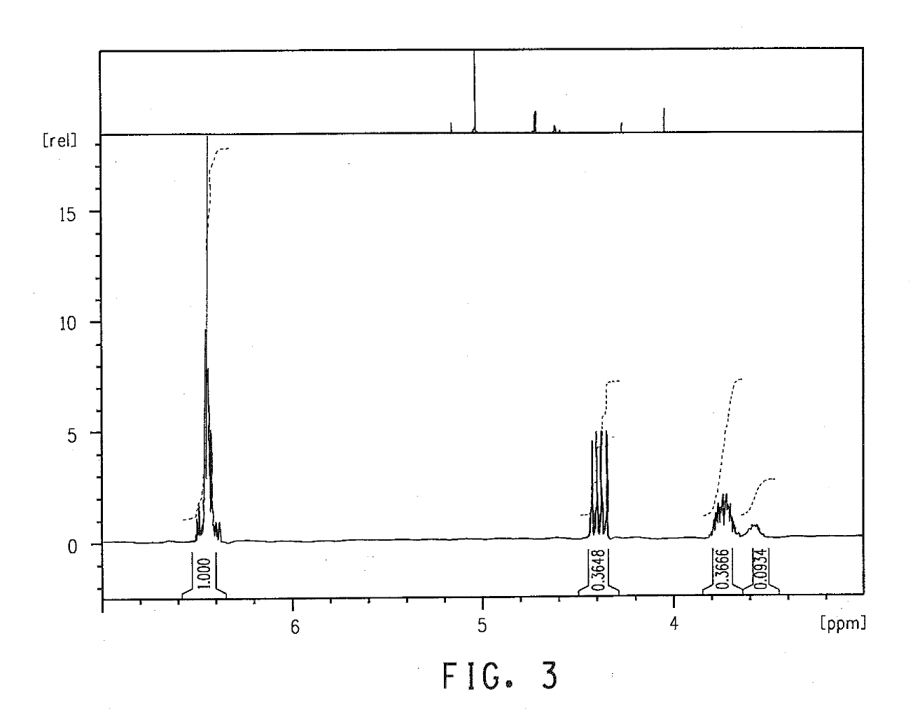

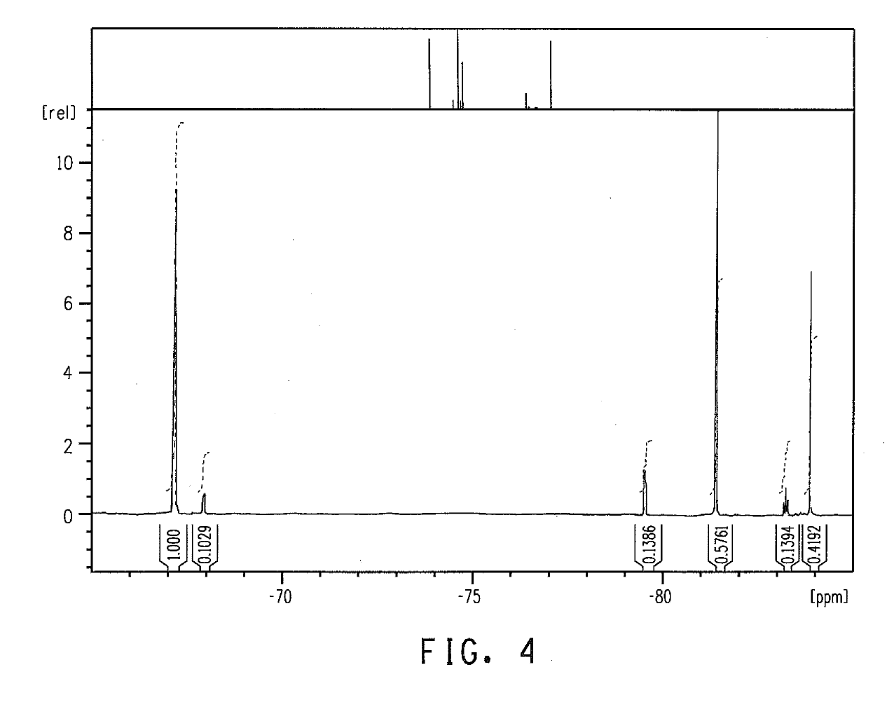

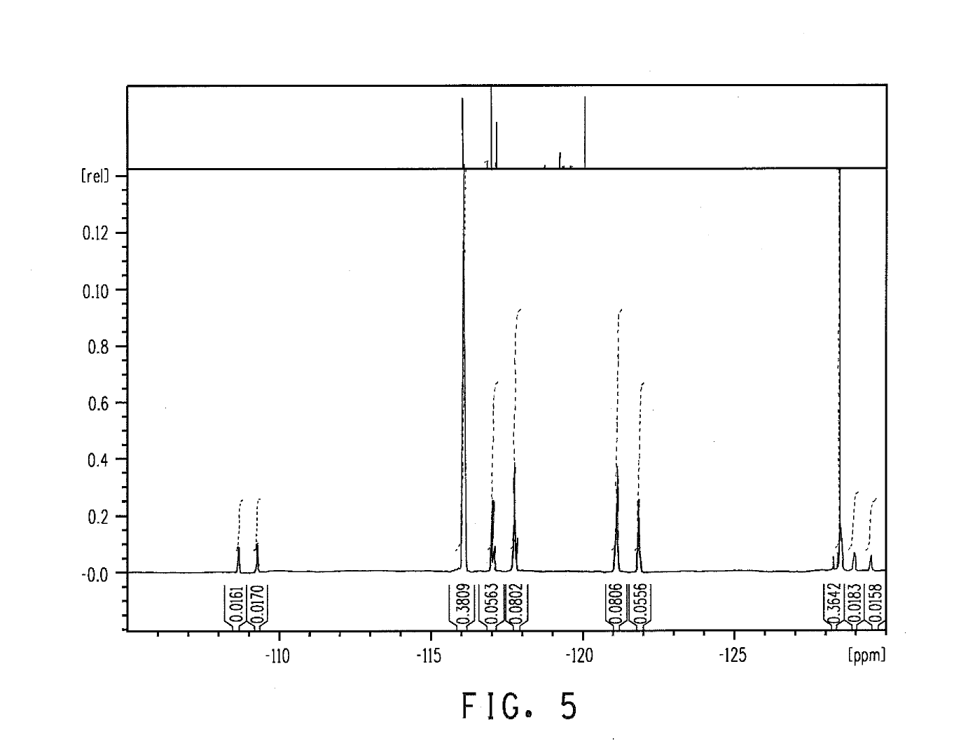

The main fraction was separated from water that was carried over in the distillation, dried over magnesium sulfate, and filtered through polypropylene into the 1-L pot of a small spinning band apparatus. The product was re-distilled and about 7-mL forerun was collected at 51.4° C. to 53.0° C. A main fraction about 600 mL, was collected in two lots (500 and 100 mL) at 53.4° C. to (53.9° C. to 54.2° C.). A GC/MS (Cryo-Mass Spectrometry method) indicated that the main product was greater than 99.9% of the previously identified three highly overlapped 153-10 isomers. The ¹H and ¹⁹F NMR spectra of the main product (500 mL lot) are shown in FIG. 3-5.

### Example 4

Example 4 demonstrates the distillation of crude 153-10 isomers from the reaction of trans 1,1,1,4,4,4-hexafluoro-2-butene with tetrafluoroethylene in presence of SbF₅ catalyst with a starting reaction temperature of −50° C.

The crude mixture of the 153-10 isomers was purified by distillation to remove remaining trans 1,1,1,4,4,4-hexafluoro-2-butene, and isolating the 153-10 isomers from higher TFE analogues (e.g. 4,5-dihydrotetradecafluoro-4-octene and/or other 173-14 isomers). The distillation apparatus consisted of a 0.5-L pot, a heating mantle with magnetic stirring; an 18-inch, Hastelloy®-packed and vacuum-jacketed column, a high-reflux-ratio (60:3 s/s) still-head with magnetic valve; and a condenser (starting at −15° C.). The crude product (236 g) was cannula-transferred to a dry-ice-chilled still-pot. A first fraction (about 54 g) of trans 1,1,1,4,4,4-hexafluoro-2-butene isomers was isolated at about 9.4° C. to 12.8° C. A second main fraction (about 153 g) was obtained at 46° C. to 52-54° C. The main product also contained 0.6% residual trans 1,1,1,4,4,4-hexafluoro-2-butene isomers. The heel (BP>73° C.) weighed about 28 g. A GC/MS (Cryo Mass-Spectrometry method) indicated that the main product was 99.4% of apparently two highly overlapped 153-10 isomers. The main product also contained 0.6% residual trans 1,1,1,4,4,4-hexafluoro-2-butene isomers. The heel contained 36% remaining 153-10's, 57% 4,5-dihydrotetradecafluoro-4-octene (173-14mcczz), and 7% other 173-14's compounds (5 peaks).

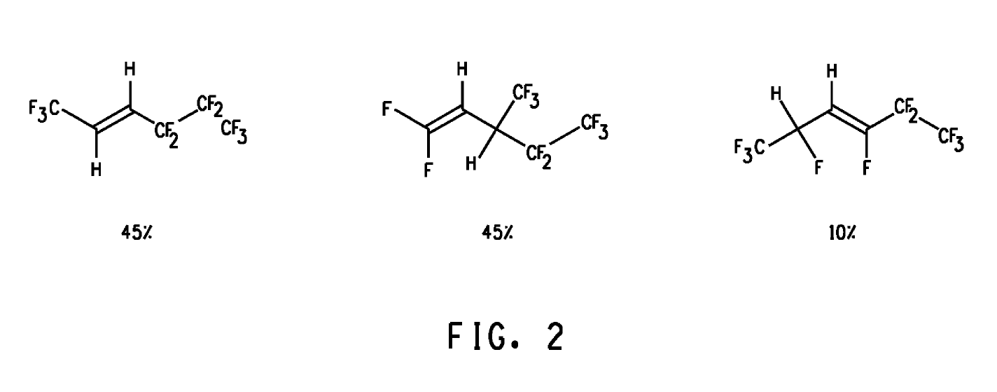

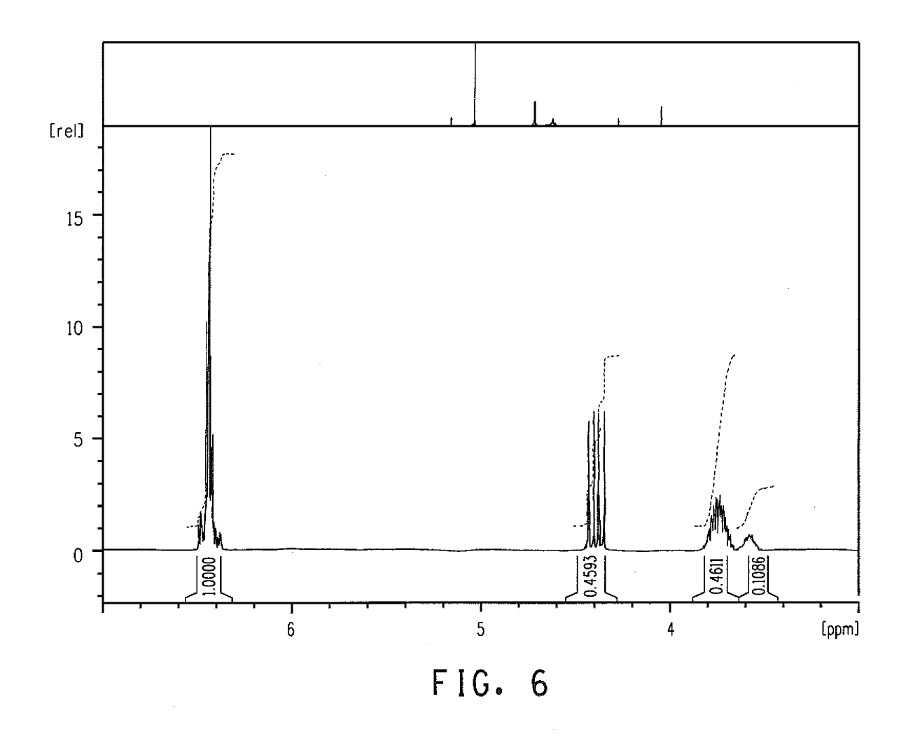

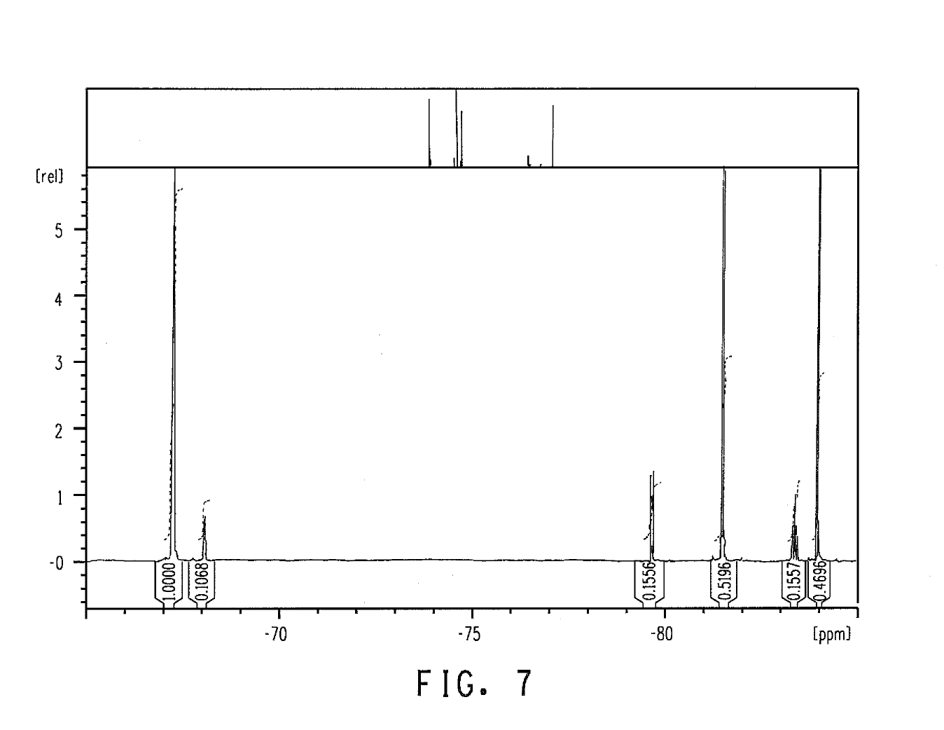

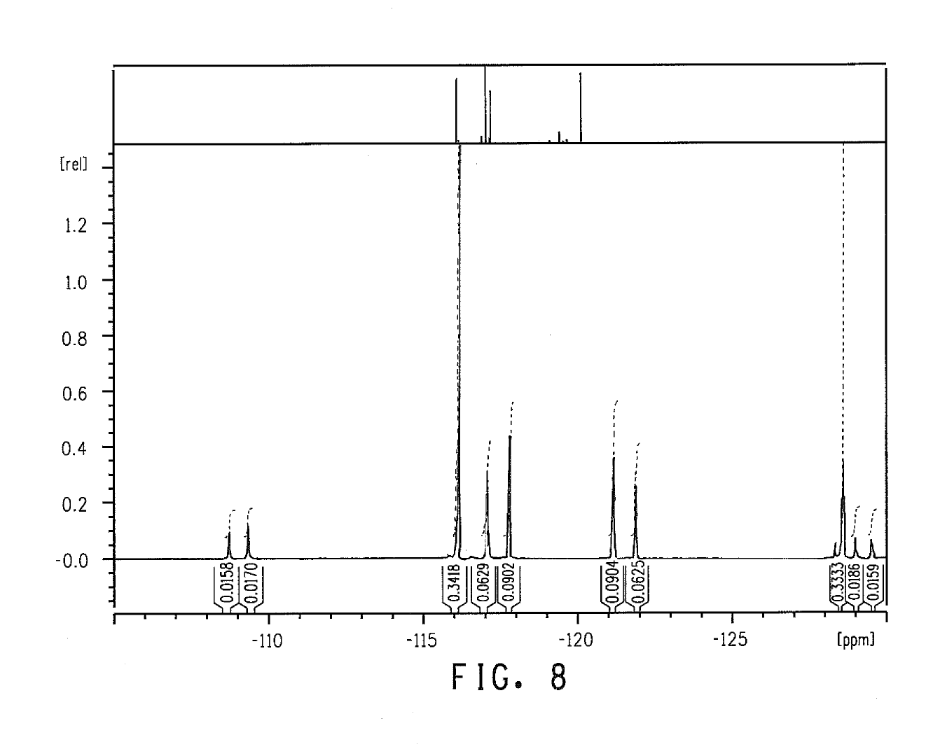

The main fraction was re-distilled using a small spinning band column. A 7-mL forerun was collected at 51.4 to 53.6° C. A main 80-mL fraction was collected at 53.6-53.9° C. A GC/MS (Cryo Mass-Spectrometry method) indicated that the main product was greater than 99.9% of three overlapped 153-10 isomers (1,1,1,4,4,5,5,6,6,6-decafluorohex-2-ene). The ¹H and ¹⁹F NMRs of the main product are shown in FIGS. 2 through 4. The suggested structures and relative concentrations of 153-10 isomers are shown in FIG. 2. The ¹H and ¹⁹F NMRs of the main product are shown in FIGS. 6 through 8.

### Example 5

Example 5 demonstrates the use of a 153-10 isomer mixture as a carrier fluid to deposit a coating of a fluorinated oil.

The ability of the 153-10 isomers to dissolve a fluorinated oil is determined by adding increasing amounts of the oil to the isomers until the mixture becomes turbid or splits into two phases. The test shows the oil is miscible in all proportions in the solvent and no turbidity is observed. This is shown in Table 3. In addition, a solution of 5 wt % of the oil is prepared in the 153-10 isomers. Pre-weighed metal coupons with a surface area of 38.5 cm² are dipped into the solution, the solvent evaporates, and the coupon is re-weighed. Table 1 shows the average of 3 coating thicknesses made with this dip coating process. Thus, 153-10 isomers can be used as a carrier fluid for the deposition of the fluorinated oil onto a substrate.

TABLE 3

| | Solubility of 153- | Coating thickness |
|---|---|---|
| oil | 10 isomers | (ave of 3) |
| Krytox GPL-106 oil | miscible | 160 ug/cm² |

### Example 6

Example 6 illustrates removing oil from a surface using a 153-10 isomer mixture.

The ability of the 153-10 isomers to clean a fluorinated oil off a substrate is determined by preparing metal coupons that are coated in Krytox GPL 106 oil and then cleaning the coupons. After coating the coupon with oil, the coupon is immersed into the 153-10 isomers at a temperature of about 50° C. for 5 minutes. The weights of the coupon before and after cleaning are measured and the % oil removed is calculated. Results in Table 4 show the ability of the solvent to remove the oil and therefore the solvent would be an effective cleaning agent.

TABLE 4

| | | Wt. coupon | Wt. coupon | |
|---|---|---|---|---|
| | Wt coupon | after | after | % oil |
| Coupon | clean (g) | coating (g) | cleaing (g) | removed |
| 1 | 10.5123 | 10.5184 | 10.5124 | 98.4 |
| 2 | 10.1354 | 10.1418 | 10.1354 | 100 |
| 3 | 10.4698 | 10.4757 | 10.4698 | 100 |

Note that not all of the activities described above in the general description or the examples are required, that a portion of a specific activity may not be required, and that one or more further activities may be performed in addition to those described. Still further, the order in which activities are listed are not necessarily the order in which they are performed.

In the foregoing specification, the concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

It is to be appreciated that certain features are, for clarity, described herein in the context of separate embodiments, may also be provided in combination in a single embodiment. Conversely, various features that are, for brevity, described in the context of a single embodiment, may also be provided separately or in any subcombination. Further, reference to values stated in ranges include each and every value within that range.

What is claimed is:

1. A composition comprising at least one or more of:
(A) a first isomer 1,1,4,4,5,5,5-heptafluoro-3-(trifluoromethyl)pent-1-ene;
(B) a second isomer 1,1,1,2,2,3,5,6,6,6,-decafluorohex-3-ene; and
(C) mixtures thereof.

2. The composition as recited in claim 1, further comprising a third isomer 1,1,1,4,4,5,5,6,6,6-decafluorohex-2-ene.

3. The composition as recited in claim 2, wherein said first isomer, said second isomer, and said third isomer, respectively, are in the range of from about:
(I) said first isomer is in the range of from about 40% to about 60%;
(II) said second isomer is in the range of from about 0 to about 15%; and
(III) said third isomer is in the range of from about 40 to about 50%,
of the total weight of said first isomer, said second isomer, and said third isomer.

4. The composition as recited in claim 3, wherein said composition comprises said first isomer and said third isomer;
wherein said first isomer is about 55% and said third isomer is about 45% by weight of the total weight of said first isomer and said third isomer; and
wherein the weight content of said second isomer is substantially zero.

5. A process for preparation of composition of claim 1, comprising:
(A) contacting 1,1,1,4,4,4-hexafluorobut-2-ene with 1,1,2,2-tetrafluoroethylene, to provide said composition mixed with other reaction products; wherein said contacting step takes place in the presence of a catalytically effective amount of:
(i) MCl_{5-y}F_{y}, wherein M=Sb, Nb, Ta, Mo, and y=0 to 5;
(ii) SbCl₃₋ₓFₓ (x=0 to 3);
(iii) aluminum halide composition having a bulk formula of AlX_{y}F_{3-y}, wherein the average value of y is 0 to 3, and wherein X is Cl or Br;
(iv) BF₃;
(v) FeX₃ wherein X is selected from the group consisting of Cl and F, and FeX₃ supported on carbon;
(vi) AsF₃; and/or
(vii) M′Cl_{4-z}F_{z}, wherein M′=Sn, Ti, Zr, Hf; z=0 to 4, and
(B) optionally separating said other reaction products from said composition.

6. The process as recited in claim 5, wherein said contacting step is undertaken in the temperature range of from about −50° C. to about +20° C.

7. The process as recited in claim 6, wherein said catalyst is SbF₅.

8. The process as recited in claim 5, wherein said contacting step is undertaken in the temperature range of from about −45° C. to about −55° C.

9. A method for removing residue from a surface of an article comprising:
(A) contacting said surface with a composition of claim 1, and
(B) recovering the surface from said composition.

10. The method as recited in claim 9, wherein said composition further comprises a propellant.

11. The method as recited in claim 10, wherein said propellant is selected from the group consisting of air, nitrogen, carbon dioxide, difluoromethane (CF₂H₂, HFC-32), trifluoromethane (CF₃H, HFC-23), difluoroethane (CHF₂CH₃, HFC-152a), trifluoroethane (CH₃CF₃, HFC-143a; or CHF₂CH₂F, HFC-143), tetrafluoroethane (CF₃CH₂F, HFC-134a; or CF₂HCF₂H, HFC-134), pentafluoroethane (CF₃CF₂H, HFC-125), 1,3,3,3-tetrafluoro-1-propene (HFO-1234ze), 2,3,3,3-tetrafluoro-1-propene (HFO-1234yf), 1,2,3,3,3-pentafluoropropene (HFO-1225ye), 1,1,3,3,3-pentafluoropropene (HFO-1225ze), hydrocarbons, and dimethyl ether.

12. The method as recited in claim 9, wherein said composition further comprises at least one surfactant.

13. The method as recited in claim 9, wherein said contacting is accomplished by vapor degreasing.

14. The method as recited in claim 13, wherein said vapor degreasing is performed by:
(A) boiling the composition; and
(B) exposing said article to vapors of the boiling cleaning composition.

15. The method as recited in claim 9, wherein said contacting is accomplished by immersing the article in said composition, wherein the composition is at a temperature greater than ambient or room temperature.

16. The method as recited in claim 15, wherein said composition is at a temperature of about the boiling point of said composition.

17. The method as recited in claim 9, wherein said contacting is accomplished by wiping the article with an object soaked in said composition.

18. A method for depositing a fluorolubricant on a surface comprising:
(A) combining a fluorolubricant and a solvent, said solvent comprising said composition of claim 1 to form a lubricant-solvent combination;
(B) contacting said combination of lubricant-solvent with the surface; and
(C) evaporating said solvent from said surface to form a fluorolubricant coating on said surface.

19. The method as recited in claim 18, wherein said surface is that of a semiconductor material, metal, metal oxide, vapor deposited carbon, or glass.

20. The method as recited in claim 19, wherein said surface is that of a magnetic medium.

21. The method as recited in claim 20, wherein said magnetic medium is a computer disk.

22. The method as recited in claim 20, wherein said contacting step is accomplished by dipping or immersing said surface in a bath comprising said fluorolubricant.

23. The method as recited in claim 18, wherein said contacting step is accomplished by spraying or spin coating said surface with said fluorolubricant.

24. The method as recited in claim 18, wherein said fluorolubricant concentration in the lubricant-solvent combination is from about 0.02 weight percent to about 0.5 weight percent.

25. The method as recited in claim 18, wherein said evaporating step is accomplished at a temperature of from about 10° C. to about 40° C.
